(19) United States
(12) Patent Application Publication
(10) Pub. No.: US 2007/0218127 A1
(43) Pub. Date: Sep. 20, 2007

(54) Capsules Derived From Asymmetrical Production

(75) Inventors: Edward Zbygniew Nowak, Cambridge (GB); Andrew John Sharp, Norfolk (GB)

Correspondence Address: MCDERMOTT WILL & EMERY LLP, 18191 VON KARMAN AVE., SUITE 500, IRVINE, CA, 92612-7108, US

(73) Assignee: Bio Progress Technology Limited, March, Cambridgeshire (GB)

(21) Appl. No.: 11/579,155

(22) PCT Filed: Jun. 3, 2005
(86) PCT No.: PCT/GB05/02174

§ 371 Date: Mar. 8, 2007

(30) Foreign Application Priority Data
Jun. 4, 2004 (GB) 0412495.4

(51) Int. Cl. A61K 9/48

(52) U.S. Cl. 424/463

(57) ABSTRACT

A method of making a capsule comprising: -forming one or more first films into a hemi-capsule, -filling the hemi-capsule with one or more capsule contents, -applying one or more second film to said hemi-capsule to form a complete capsule, said one or more second film being flat prior to capsule formation, -a curing stage to alter the shape of the capsule.

### FIELD OF INVENTION

This invention relates to a process of producing shaped soft capsules from asymmetrical shaped capsules, which may be termed as half capsules or hemi-capsules. The hemi-capsules may be produced from water soluble, thermoplastic polymer films such as those cast from modified cellulose ether solutions.

### SUMMARY OF INVENTION

Hemi-capsules can be prepared by thermoforming and/or vacuum forming a single sheet of film into a desired shape, usually within a recess. This ‘cup’ shape can then be filled with a suitable material to provide the contents of the capsule, such as a medicament, drug, cosmetic or foodstuff. The filled cup-shape is then lidded with film. Another single sheet of film of the same/similar or different material as the previous formed film may be used. The lidded filled cup may then be sealed e.g. along its perimeter edge. Suitable methods of sealing can include the use of heat, radio frequency, ultrasonics, glues or lasers, or indeed any combination of the above. The aforementioned process can result in a suitably filled capsule, half capsule or hemi-capsule which may be distinctly asymmetrical, distinctly symmetrical or indeed may be at some point inbetween these extremes.

The hemi-capsules according to the present invention can be placed, for a pre-determined period of time, in a humid environment, moisture laden medium or heated (e.g. placed in a hot stream of air) Such an environment may or may not be heated to hydrate and raise the moisture level within the film, rendering a previously rigid film flexible. This may result in a change of capsule shape e.g where the thermoformed side of the capsule film can have a memory, e.g. whereby it may attempt to move back to its original state (for example, in this case, a flatter, unformed film) and the lidding film moves outwards to compensate, for example, it may move outwards, or bulge, as the other film attempts to return to its original shape. The result can be that a capsule tends towards becoming symmetrical and may resembles a typical softgel capsule in its overall shape. The capsule can then be ‘cured’ by placing it into a e.g reasonably dry atmosphere where its shape may be retained and it may resemble a ‘typical softgel capsule.

Many types of polymeric film may be suitably used in the process according to the present invention. Thermoplastic and/or water soluble films, which can have a ‘memory’, can be suitable for this process. Ideally suited films are modified cellulose films such as the family of cellulose ether polymers. Suitable polymers according to the present invention include types of polymers which are or tend to be water soluble and/or thermoformable, and may include hydroxypropylmethyl cellulose (HPMC), hydroxypropyl cellulose (HPC), hydroxyethylmethyl cellulose (HEMC), hydroxyethyl cellulose (HEC), or methyl cellulose (MC), but the invention is not restricted to these polymers. Other polymers can include polyvinyl alcohol (PVOH) and polyethylene oxide (PEO).

There are a number of benefits of forming capsules in the manner according to the present invention, as they can be made on simplified machinery, produced more quickly and be more cost effective to manufacture. The quality of the capsule provided in accordance with the present invention, is more consistent, as compared with current comparable methods of producing same. Also, utilizing the process according to the present invention, there is less waste due to capsule defects and the capsules produced can have a consistently higher seam burst strength e.g. as compared to capsules of a similar size and/or shape, for example, made using a process by which two identical capsule halves are bonded/welded together.

GB 617,723 discloses a method of producing double chamber gelatin capsules by bringing two flat sided gelatin capsules together. However, the physical nature of gelatin results in capsules and a capsule formation process that are both very different in character/nature to that presently described.

### DETAILED DESCRIPTION OF THE INVENTION

The process of making full shaped symmetrical capsules from hemi capsules can be dependant on:
1) The conditions the capsules are initially subjected to. (e.g. temperature and humidity).
2) The thickness of the film used in the production of the two halves of the capsule.
3) The plasticisers used in the film formulation itself.
4) The polymeric material used to produce the film.

It has been found, that for particular purposes, the most suitable polymeric films are water soluble, such as those found in the cellulose ether family (e.g. HPMC), as this can be cast from aqueous solutions and dried to produce an even thickness film.

Certain plasticers used within a cellulose ether film will make the film easier to thermoform into suitable shapes and render the film more elastic. Suitable plasticisers, which could be used in food and drug applications are:-glycols, carboxylic acids, acetins and citrate esters, for example, glycerin, propylene glycol, citric acid, lactic acid, mono, di and Triacetin, Triethyl citrate, tributyl citrate. The most appropriate plasticisers for this invention, include those which have a strong affinity for water, as this can, for example, can enable the film to hydrate more quickly.

Film thickness can also affect the rate of capsule shape change. To enable rapid changes to take effect, e.g. during the ‘curing’ of the capsules, the ‘lidding’ film could be up to 50% thinner than the ‘thermoformed’ film. For example if a film of 125 microns is used as the film, to form a cavitated shape and thermoformed into a capsule half, the lidding film, ideally, could be 80 microns thick, as this allows for an easier transition from the hemi to full capsule shape.

Ideally, according to one aspect of the present invention, the best humidity currently found to be suitable to subject the capsules to is between 60-90% R.H. and preferably between 75-85% R.H. The capsules can be maintained at temperatures of between 20-30° C. with little risk of any decomposition of the contents, and this in addition can accelerate the process of shape change.

Finally the capsules can be dried to reduce the capsule shell moisture level.

In an aspect of the present invention, after capsule formation, the capsule can retain its original shape (as at the instant of formation), even after the capsule is released and is free to assume its own natural shape in free space. To further exemplify this aspect of the invention, the capsule e.g. can still retain the original formed shape, or similar, and only after it is subjected to further processing, does it begin to change shape e.g. tend toward being a more symmetrical capsule. Such further processing may involve heat and/or humidity.

In another embodiment of the present invention, a liquid composition—e.g. a glue, adhesive or hydrating fluid is used to assist in the formation of a capsule. The liquid composition can be applied to the surface of the film(s) before they make contact with each other during the process of capsule formation. The liquid composition may comprise:
1) one or more polymers
2) one or more organic compounds
3) water

In a preferred embodiment of this aspect of the invention, the one or more polymers may be the same or similar polymers to that which form the film to which the liquid composition it to be applied to, e.g. HPMC, or other polymers hereinbefore described. The organic compounds(s) may include organic acids, e.g. carboxylic acids such as fruit acids/alphahydroxy acids. The acids may include lactic acid, citric acid, malic acid, glycolic acid or tartaric acid. These organic compound may also be incorporated into the film compositions.

In certain embodiments of the invention, it may be advantageous for the capsule to retain a flat or relatively flat side, so that it can be stored more conveniently and take less space (the flat caps may be flipped in alternate orientations to be more compactly stored. Flat caps may retain their shape by being kept at e.g. 20 degrees Celsius and 50% relative humidity. Generally lower temperatures and lower humidities can favour original shape retention. There may be other reasons why a flat shape is required, e.g. so that the capsule can be placed on a flat or relatively flat surface, where it is requires to remain in position (and not roll e.g. on the tongue or skin). The capsule may then release its contents in an advantageous way. The capsule may be so designed that it cannot be swallowed or it is difficult to swallow. The capsule may be so designed that it (inevitably) releases it's contents in the mouth. The flat nature of the side of the capsule also has advantages in respect of the contact area it may present to a surface. Higher degrees of surface contact may result in the more accurate application of active ingredients to or through a surface, even before the (complete) destruction of the capsule; in these embodiments of the invention, a capsule with a thinner or more soluble ‘flat side’ may be appropriately designed for a particular application, such that e.g. a capsule at the site of delivery, may sit on a surface on the capsule's ‘flat’ side, and may deliver its active ingredients only through the area of contact (e.g. the flat side of the capsule), such that e.g that flat side may be preferentially degraded or destroyed, perhaps resulting in the contents of the capsule being in direct contact with the surface and e.g. having the effect of beneficial absorption or controlled or accurate application of actives to the surface of e.g an organ of the body. Additionally, the ‘flat cap’ may be so designed as to rest on or stick to a particular surface and, e.g. be moisture adhesive or muco-adhesive, so that the capsule remains in the initial position where it was first applied or placed. Films used in accordance with the present invention may conveniently posses the above properties

### EXAMPLE 1

(Process 1):

Preparation of the Hemi-Capsules

1) Thermoformable film is placed over a shaped cavity, heated and vacuum formed to take the exact cavity shape.

2) The cavity containing the film is fully dosed with a liquid medicament.

3) A lidding film of the same material used in (1) but 30% thinner gauge, is placed onto the filled cavity and heat welded along the perimeter of the cavity to form the hemi-capsule (any trapped air in the capsule can be dispelled at the point of lidding).

Conditioning of the Hemi-Capsules

1) The hemi-capsules are cut out of the waste film web, placed on trays and moved into a high humidity environment of 75% to 90% R.H. The temperature should also be maintained between 20-30° C.

2) Within 24 hours the capsule shell with have hydrated sufficiently for it to become elastic and take on the appearance of a symmetrical shaped soft capsule

3) the soft capsules are then transferred into a drying environment eg:—40% R.H. 25-30° C. After 30 minutes the capsules are firm enough to be handled and have taken a shape that resembles symmetrical capsules.

The following further examples demonstrate the process.

A) Film compositions tested.

TABLE 1

Film Formula No

| Component | 1 | 2 | 3 | 4 |
|---|---|---|---|---|
| HPMC* | 77 | 77 | 80 | 80 |
| Glycerin | | 3 | 10 | 10 |
| Citric Acid | | 20 | | |
| Lactic Acid** | | | | 10 |
| Triethyl citrate | | | 10 | |
| Monoacetin | 23 | | | |

*Methocel E50 LV Premium, ex Dow Chemicals.
**Lactic acid in the form of an 80% aqueous solution.

Each film (1-4) was cast from an aqueous solution and dried in warm air conditions to provide 2 film thicknesses of 125 microns and 85 microns. The 85 micron film being used as the lidding film.

Hemi-capsules were made and filled with a technical white oil (kristol M14, ex Carless). Prior to forming and filling the capsules each half of the film used was internally coated with a hydrating solution to aid in the heat sealing of the film and to accelerate the hydration of the capsule.

The hydrating fluid used was of the following composition:

| | % w/w |
|---|---|
| HPMC (methocel E50 LV Premium) | 5.0 |
| Citric Acid | 45.0 |
| Purified water | 50.0 |

This was applied on both thicknesses of film (85 and 125 microns) at a rate of 30 grams per sq. metre.

Once the hemi-capsules were heat sealed they were placed in conditioning environments and observed for shape change and ovalisation over 24 hours.

Results.

A) Conditioning capsules at 30° C., 75% RH.

TABLE 2

| Film | | | |
|---|---|---|---|
| Formula No. | 2 hours | 4 hours | 24 hours |
| 1 | 30% ovalised | 60% ovalised | Fully ovalised |
| 2 | 50% ovalised | 80% ovalised | Fully ovalised |
| 3 | 10% ovalised | 20% ovalised | 90% ovalised |
| 4 | No change | No change | 10% ovalised |

B) Conditioning capsules at 22° C., 85% RH.

TABLE 3

| Film | | | |
|---|---|---|---|
| Formula No. | 2 hours | 4 hours | 24 hours |
| 1 | 50% ovalised | 90% ovalised | Fully ovalised |
| 2 | 50% ovalised | 90% ovalised | Fully ovalised |
| 3 | No change | 50% ovalised | Fully ovalised |
| 4 | No change | 20% ovalised | 90% ovalised |

### EXAMPLE 2

(Process 2):

These alternate processes produces rapid changes in shape (within a few seconds) compared to Process 1.

Preparation of the Hemi-Capsules.

This is identical to that described in Process 1. In addition, to aid the transformation in shape, the capsules can be filled with a liquid formulation which contains a small percentage of water, up to 5%. This water ideally (but not necessarily) should be associated with and bound to a solution, for example a salt solution or carbohydrate solution such as sorbitol. One reason for using ‘bound’ water or water based solutions, is to control the movement of water through the capsule shell. Using only free, unbound water may well result in the capsule ‘self destructing’.The careful selection of what water solution is used, or how the water is bound can be an important factor in the production of the final capsule (shape). Indeed, the ultimate shape of the capsule may be engineered by adjusting the amounts of water in a capsule, and also augmenting the way in which it is bound.

A bi-chamber capsule can be produced by introducing the flat sides of 2 hemi spherical capsules together. Each chamber of the capsule thus produced, may contain different materials, such as liquids, powders (loose or compressed/compacted), waxes or pastes, and/or different actives.

Conditioning of the Hemi-Capsules (Process 3)

The capsules are placed under a draft of hot air at a temperature in excess of 100° C. until the shape change occurs (normally 2 seconds).

Conditioning of the Hemi-Capsules (Process 4)

The capsules are immersed into a hot liquid, gel, suspension or paste which contains water, bound to a salt or carbohydrate solution where there is very little free water. The heated mass needs to be at 80° to 120° C. for the capsules to experience a rapid deformation. In this situation hemi-capsules form symmetrical capsules usually within 1-2 seconds of immersion.

The product produces according to the present invention can have specific uses, e.g. in the pharmaceutical/medicinal, VMS, food/confectionary, (such as a jam, jelly or paste), or agrochemical areas.

### DESCRIPTION OF THE DRAWINGS

The following description is intended in no way to limit the scope of the invention.

FIG. 1 shows a first film (1) over a capsule forming recess (2), and vacuum forming channels (7).

FIG. 2 shows the first film (1) drawn into a capsule forming recess (2)

FIG. 3 show capsule contents (3) being introduced into the film lines recess and

FIG. 4 shown film lined recess filled to the top/completely filled with capsule contents (3).

FIG. 5 shows a second film (4) above a film lined recess filled up to the ‘level’ and

FIG. 6 shows a second film (4) laid flat on the surface of the filling which has filled the film lined capsule recess.

FIG. 7 shows a compression and/or heating and or/sealing stage, using sealing tool (5), after which descrete capsules e.g. completely enclosing the capsule contents from the exterior, are formed. These capsules, at this point may be e.g. trapped in a film web.

FIG. 8 shows a cutting tool (6) approaching the films at the periphery of the film lined recess and FIGS. 9 and 10 show cutting tool (6) cutting through the first and second films respectively.

FIG. 11 shows a resulting semi circular or flat sided capsule (with flanges) which has been released from the capsule forming recess, (and is free standing on a surface) and FIG. 12 shows the same capsule as in FIG. 11, but after time (x): that is, a substantially symmetrical capsule.

FIG. 13 shows a capsule forming process ‘in the die’ and FIG. 14 shows the subsequent capsule forming process, ‘out of the die’ .

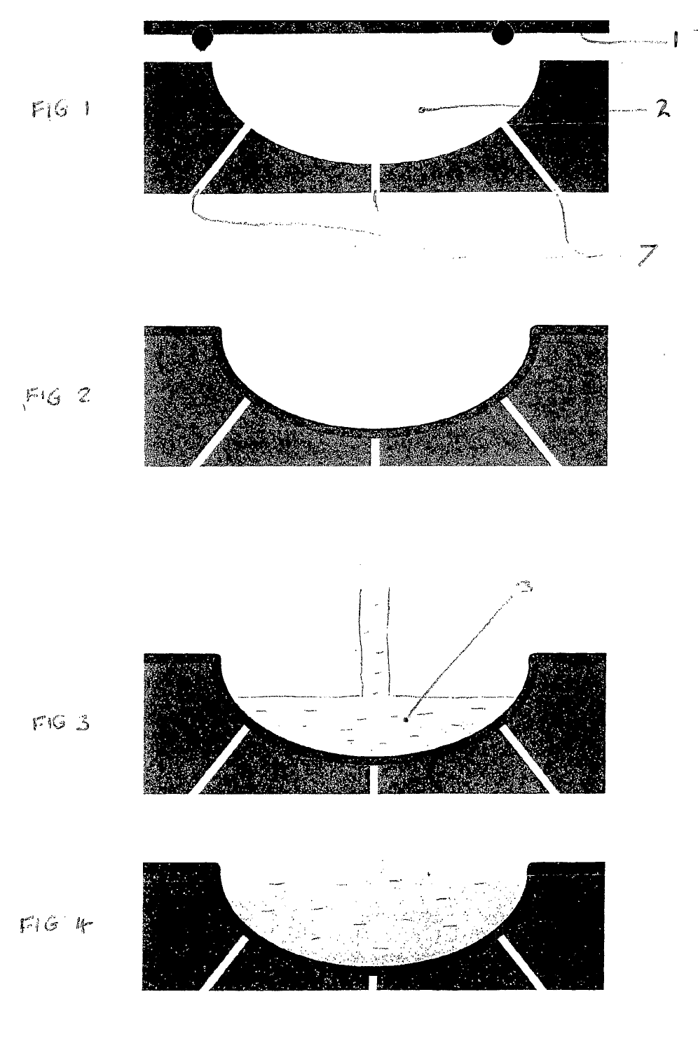

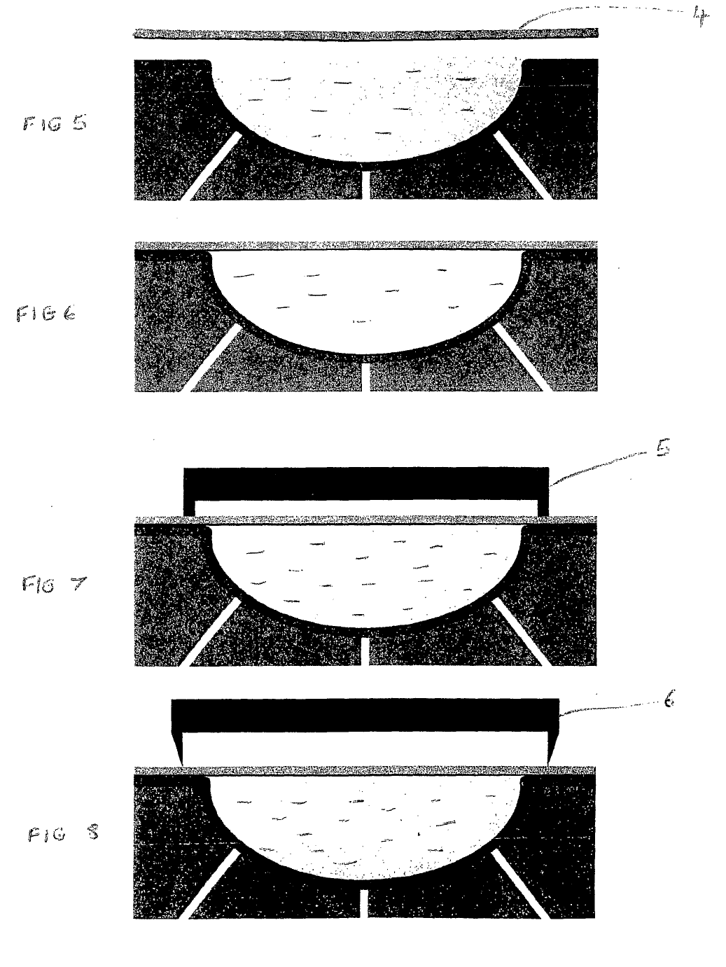

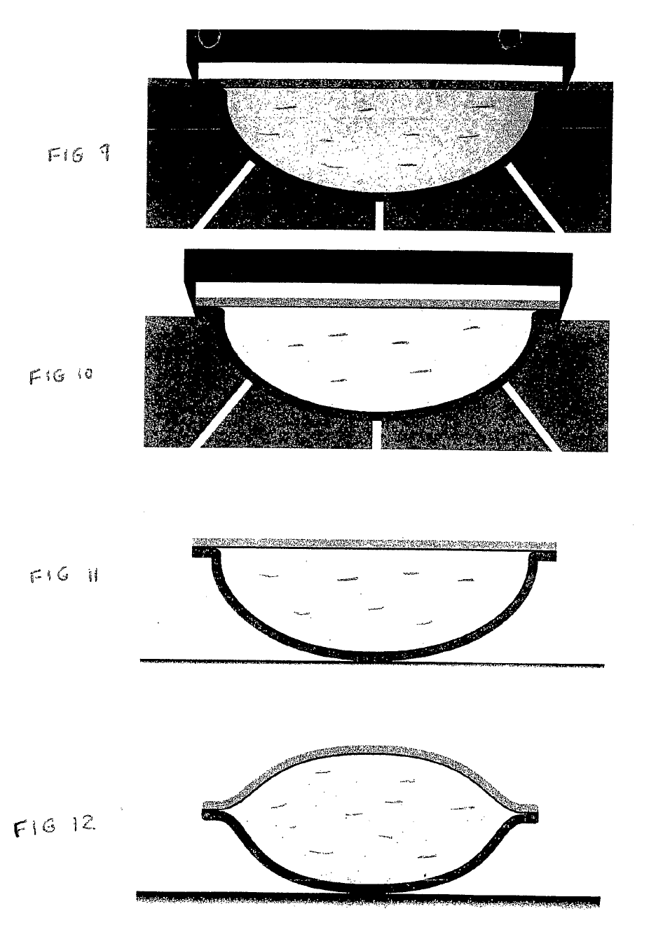

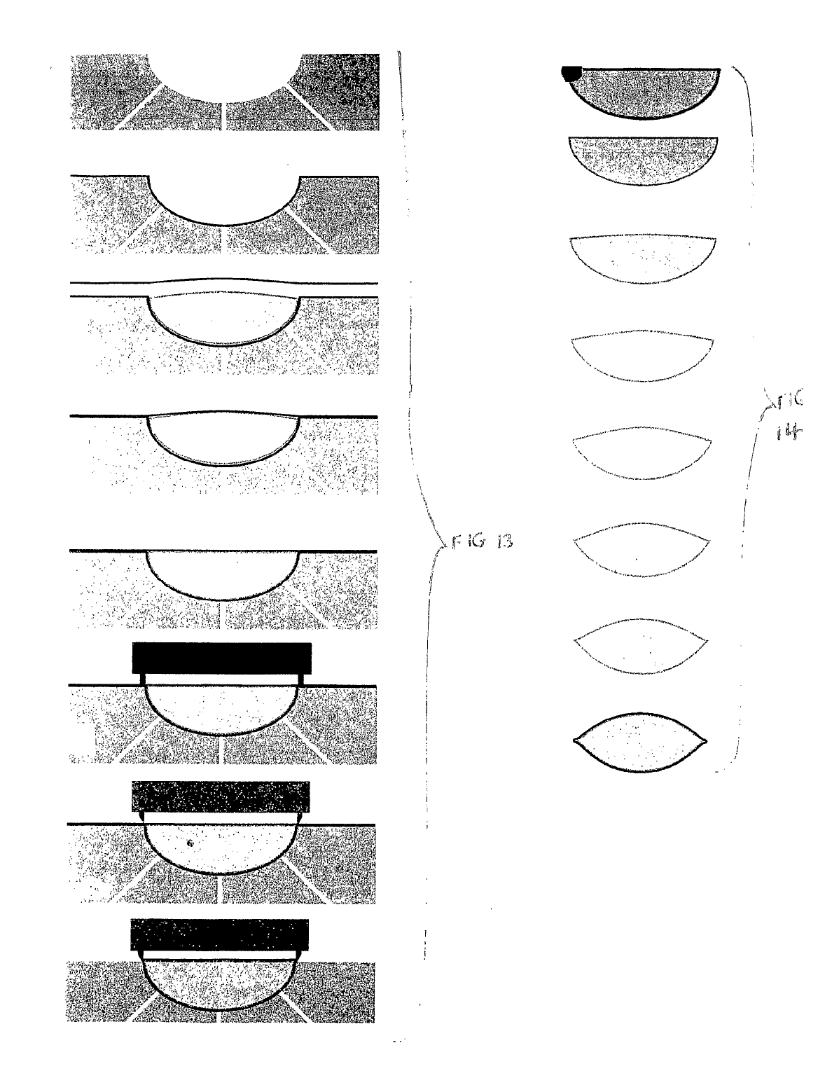

1. A method of making a capsule comprising:
a) Forming one or more first films into a part capsule shape or shapes
b) Applying one or more second films to said part capsule shape or shapes to form a complete capsule or capsules, wherein, prior to capsule formation, it is not necessary to form the one or more film(s) into a pre-formed shape.

2. A method according to claim 1 wherein, prior to capsule formation, said second film or films is flat or substantially flat

3. A method according to claim 1 or 2 wherein the one or both of the first and second film(s) comprise one or more non gelatin film forming polymers.

4. A method according to claim 3 where the film(s) are water soluble, ingestible and/or biodegradable.

5. A method according to any previous claim wherein the joining of the one or more first or second films is assisted by pressure and/or heat and/or adhesive

6. A method according to any previous claim wherein the shape of the capsule formed, when released from a capsule shape forming element, does not change or substantially change

7. A method according to claim 5 wherein the shape of the capsule does not change at the instant of release, or soon after the release from the capsule shape forming element.

8. A method according to claim 5 wherein the shape of the capsule does not change at the instant of release, or soon after the release from the capsule shape forming element, without further external influence.

9. A method according to any previous claim, but additionally including a ‘curing’ stage to alter the shape of the capsule

10. A method according to any previous claim which also includes a ‘curing’ stage to improve the strength and/or appearance of the capsule

11. A method according to any previous claim wherein said method also comprises an additional stage of filling said part capsule with one or more capsule contents to form a partly or fully filled part capsule.

12. A method according to any previous claim wherein a capsule with one substantially flat face is produced

13. A method according to any previous claim wherein substantially half the complete capsule produced is substantially flat or resembles a flat ovoid shape and the other half of the complete capsule is more rounded

14. A method according to any previous claim wherein a capsule incorporating a substantially flat face is produced immediately after complete capsule formation

15. A method according to any previous claim wherein, after complete capsule formation, the capsule deforms to a substantial degree, without mechanical assistance.

16. A method according to any previous claim, wherein the capsule formed is substantially symmetrical

17. A method according to any previous claim wherein the capsule is substantially symmetrical and the capsule seam is asymmetrical

18. A capsule produced in accordance with the method of any previous claim

19. A pharmaceutical or medical capsule according to claim 17

20. A capsule for cosmetic purposes according to claim 17

21. A capsule suitable for use in connection with agrochemicals in accordance with claim 20.

22. A multi-chamber capsule produced in accordance with any previous claim

23. A spherical bi-chamber capsule produced in accordance with any previous claim

24. A method of orally introducing a capsule in accordance with any previous claim wherein one or more active ingredients are released from the capsule and absorbed into the buccal tissues.
